(19) United States
(12) Patent Application Publication
(10) Pub. No.: US 2004/0180090 A1
(43) Pub. Date: Sep. 16, 2004

(54) Treatment of macular degeneration

(76) Inventor: Peter DeMarco, Clementon, NJ (US)

Correspondence Address: DUANE MORRIS, LLP, IP DEPARTMENT, ONE LIBERTY PLACE, PHILADELPHIA, PA, 19103-7396, US

(21) Appl. No.: 10/386,610

(22) Filed: Mar. 12, 2003

(51) Int. Cl. A61K 31/715; A61K 31/525; A61K 31/51

(52) U.S. Cl. 424/486; 514/276; 514/537; 514/54; 514/251; 514/308

(57) ABSTRACT

Methods and compositions are provided for the effective treatment of macular degeneration. The methods relate to administering a treatment including an effective amount of a combination of polyvinyl pyrollidone, procaine and thiamine to a mammalian host.

### FIELD OF THE INVENTION

Methods and compositions for the treatment of macular degenerative diseases are disclosed.

### BACKGROUND OF THE INVENTION

Age related macular degeneration (AMD) is the leading cause of irreversible central vision loss (20/200 or worse) among people in the United States aged 52 or older. AMD in the most common overall cause of blindness in the United States. AMD is a degenerative disease of the macula, the area of the retina that is responsible for central vision and color perception. AMD tends to become worse with time and can best be described as a process of “wear and tear.” Thus, the prevalence of severe visual loss increases with age. AMD encompasses several types of abnormalities that develop in the macula of older people. These abnormalities range from mild, with no loss of vision, to severe, with loss of all straight-ahead vision. Because the peripheral retina is unaffected by AMD, side vision is retained along with the ability to see in the dark. Most affected is the ability to see fine detail, to read, and to see well enough in the distance to drive.

The macula is the portion of the retina which lies directly behind the lens. The cones, light-sensitive cells which are responsible for central vision, are heavily concentrated in the macula. The peripheral retina is composed mainly of rods, the light-sensitive cells responsible for side and night vision. The macula is one hundred times more sensitive to detail that the peripheral retina. In a healthy macula, the clear layer of the retina on the inside of the eye is nourished and maintained by an adjoining layer called the pigment epithelium. Behind the pigment epithelium is the choroid which contains the blood vessels that transport nourishment to and carry waste material away from the retina.

There are three major forms of macular degeneration: dry (also known as atrophic), wet (also known as disciform, exudative, or neovascular), and pigment epithelial detachment. The dry form, which occurs in more than 85% of AMD patients, leads to gradual vision loss and can be a precursor to the wet form. The dry form results from an inability of the pigment epithelium to digest the cone tips that the retina produces as waste materials. The pigment epithelium may swell and die as a result of the collection of undigested waste materials. An early warning sign of dry macular degeneration is the formation of yellow spots, termed drusen, on the retina which result from the collection of undigested waste materials.

The wet form of macular degeneration, which occurs in about 10% of AMD patients, is associated with a sudden vision loss, resulting from the growth of new, abnormal blood vessels, also termed subretinal (choroidal) neovascularization (SRNV), under the pigment epithelium of the retina. The fluid and blood that leak from these new blood vessels cause the macula to bulge, resulting in distorted vision. As the disease progresses the cones are flooded and separated from their source of nourishment. Without nourishment, the cones degenerate and die, causing permanent blind spots. Pigment epithelial detachment, the third form of macular degeneration, may result with continued SRNV beneath the pigment epithelium, forcing it to detach from the choroid. Pigment epithelial detachment occurs in less than 5% of AMD patients.

Drusen, although used as an indicator of the development of macular degeneration, are currently not treated. Instead, patients with drusen are closely monitored through regular eye exams. At present there is no therapeutic or surgical treatment for the dry form of AMD. However, the dry form of AMD is accompanied by only gradual vision loss and the concomitant damage is usually not as severe, nor as sudden as, the damage associated with the wet form of AMD. Eyesight in patients with the dry form of AMD may be helped by special low vision spectacles or by learning to use side vision to accommodate for the loss of central vision. Because the dry form can lead to the wet form, patients are encouraged to monitor their vision using the Amsler Grid. This simple test can detect early retinal deformation caused by neovascularization associated with the wet form of macular degeneration.

At present there is no cure for AMD. The only treatment available for SRNV associated with the wet form of macular degeneration is laser photocoagulation. A narrow, highly focused beam of laser light is directed at the abnormal blood vessels. The heat produced by the laser dries up leaking blood vessels and inhibits further leakage, bleeding, and growth. However, laser treatment is effective only in selected classic cases, which include only about 25% of wet form patients. Furthermore, patients treated with laser photocoagulation suffer a rapid and high rate of recurrence (10% within two months, 53% within three years following treatment). Laser treatment is also quite destructive and may result in irreversible damage to the retina. No therapeutic treatments are available for treating AMD, although current research has identified several drug candidates including: growth hormones, such as basic fibroblast growth factor (bFGF) and transforming growth factor .beta.(TGF-.beta.); neurotrophic factors, such as brain-derived neurotrophic factor (BDNF); regulators of neovascularization, such as plasminogen activator factor type 2 (PAI-2); anti-inflammatory drugs, such as dexamethasone; antioxidants; and forms of interferon, such as .alpha.-2a interferon.

Certain macromolecular compounds have properties that allow them to function as antigens. The antigens as set forth in group 1 below are t-cell independent antigens, as opposed to most antigens that need t-cells to process them. Compounds from group 2 and 3 may be thought of as haptens, i.e., incomplete antigens, unless coupled to a carrier. Compounds from group 4 activate the alternative complement system. Most antigens need t-cells to help process the antigen, so that antibodies may be manufactured.

### SUMMARY OF THE INVENTION

Methods and compositions are provided for the effective treatment of macular degeneration. The methods relate to administering a treatment including an effective amount of a combination of polyvinyl pyrollidone, procaine and thiamine to a mammalian host.

### DESCRIPTION OF THE PREFERRED EMBODIMENTS

Macular degeneration is treated by the intramuscular introduction of a drug shown to be effective in the inhibition of subretinal neovascularization. Introduction of the drug allows diffusion of the drug throughout the vitreous within the posterior segment (or in the case of patients who have undergone vitrectomy, the cavity or space occupying the posterior segment) and further into the entire retina, the choroid and the opposed sclera. Thus, the drug will be directly available at the macula, the site where the drug is needed, and will be maintained at an effective dosage. The treatment was administered intramuscularly in either the triceps or gluteal regions.

The wet form of macular degeneration and pigment epithelial detachment are forms of AMD which are amenable to treatment using the method of the subject invention. In both of these forms of macular degeneration, the disease is associated with SRNV. The drugs of choice in the method of the subject invention include combinations of the various groups:

The first group includes macromolecular compounds that may be selected from the following:

a) polyvinyl pyrollidone (available as Kollidon™ from BASF, or Plasdone C from GAF Corporation), b) pneumococcal polysaccharides, or c) lipopolysaccharides (group 1);

The second group includes the salts of lidocaine, chloroprocaine, tetracaine, procaine or piperocaine (group 2);

The third group includes the salts thiamine, riboflavine, papaverine, papaveraldine, paveril, D-biotin or D-biotin in esterified or salt form (group 3);

The fourth group includes inulin or zymosan (group 4). Further details concerning various components of the present invention may be found in U.S. Pat. No. 4,618,490, incorporated herein by reference.

In the testing of the present invention, a study was conducted in order to determine therapeutic effectiveness of the respective treatments. A scale of 0.0 to 1.0 was used, in which 0.0 represented the retinal cells as not being responsive to light. Proceeding up the scale to 1.0 (“normal” vision), objects not previously visible went from an indistinct outline to a normal shape and size.

The patients treated were in their sixties, and were diagnosed as having macular degeneration for varied periods of time, i.e., from 2 to 7 years. Compositions of representative solutions administered and relative weight percentages of components are listed as A-C, below:

A

| | |
|---|---|
| Procaine HCL | 5% |
| Polyvinyl Pyrollidone (PVP) | 10% |
| Thiamine HCL | 5% |
| Papaverine HCL | 1% |
| Inulin | 1% |
| D-biotin | 0.04% |
| Chlorobutonal | 1% |
| Water q.s. | |

B

| | |
|---|---|
| Chloroprocaine | 4% |
| PVP | 12% |
| Thiamine mononitrate | 3% |
| Paveril | 0.5% |
| Chlorobutonal | 0.3% |
| Water q.s. | |

C

| | |
|---|---|
| Procaine HCL | 5% |
| PVP | 10% |
| Thiamine HCL | 2.0% |
| Riboflavine | 1.0% |
| Inulin | 0.5% |
| Chlorobutonal | 0.3% |
| Water q.s. | |

Intramuscular (IM) injections of solution A of about 10 ml daily for 10 days and thereafter about 5-10 ml twice per week took place until satisfactory results occurred. Note that if pH adjustment is necessary, it is adjusted to be compatible with IM injections into living tissues.

Results are shown in Table I, below:

TABLE I

Visual Acuity

| Patient | Dose(cc) | Before Treatment | After Treatment |
|---|---|---|---|
| 1 | 8.5 | 0.2 | 0.4 |
| 2 | 8.5 | 0.7 | 1.0 |
| 3 | 7 | 0.7 | 0.8 |
| 4 | 9.7 | 0.6 | 0.8 |
| 5 | 10 | 0.1 | 0.1 |

As shown above, patients' conditions were in general significantly improved by the treatment of the present invention.

In further preferred embodiments, the molecular weight of PVP may vary from 5,000 to 50,000. In addition, such injections may contain from about 1% to 14% (by weight) from group 1, from about 0.5% to 8% from group 2, from about 0.5% to 5% from group 3, and from about 0.01% to 1% from group 4, as noted above.

While the present invention has been described with respect to particular embodiments thereof, it is apparent that numerous other forms and modifications of the invention will be obvious to those skilled in the art. The appended claims and the present invention generally should be construed to cover all such obvious forms and modifications which are within the true spirit and scope of the present invention.

What is claimed is:

1. A method of treatment of macular degeneration in a mammalian host, said method comprising:
administering a treatment comprising an effective amount of a combination of polyvinyl pyrollidone (PVP), procaine and thiamine to said host, wherein the molecular weight of the PVP is from about 5,000 to 50,000.

2. The method as recited in claim 1, wherein said combination further comprises a compound selected from the group consisting of papaverine, inulin, paveril and riboflavine.

3. The method as recited in claim 2, wherein said combination further comprises chlorobutonal.

4. The method as recited in claim 1, wherein said treatment is administered intramuscularly.

5. A composition for treating macular degeneration in a mammalian host, comprising a combination of polyvinyl pyrollidone (PVP), procaine and thiamine, wherein the molecular weight of the PVP is from about 5,000 to 50,000.

6. The composition as recited in claim 5, wherein said combination further comprises a compound selected from the group consisting of papaverine, inulin, paveril and riboflavine.

7. The composition as recited in claim 6, wherein said combination further comprises chlorobutonal.
